(19) United States
(12) Patent Application Publication
(10) Pub. No.: US 2014/0127741 A1
(43) Pub. Date: May 8, 2014

(54) METHOD OF COLLECTION, CLASSIFICATION AND PRESERVATION OF SAMPLES CONTAINING STEM CELLS

(71) Applicants: Federico Maggi, Colazza (IT); Marco Giovanni Reguzzoni, Busto Arsizio (IT); Giuseppe Simoni, Pavia (IT)

(72) Inventors: Federico Maggi, Colazza (IT); Marco Giovanni Reguzzoni, Busto Arsizio (IT); Giuseppe Simoni, Pavia (IT)

(73) Assignee: BIOCELL CENTER S.P.A., 21052 BUSTO ARSIZIO (VARESE) (IT)

(21) Appl. No.: 14/111,309

(22) PCT Filed: Apr. 13, 2011
(86) PCT No.: PCT/IT11/00114

§ 371 Date: Dec. 10, 2013

(51) Int. Cl. G01N 33/50

(52) U.S. Cl. CPC G01N 33/5005
USPC 435/30

(57) ABSTRACT

A method of collection, classification and conservation of stem cells comprises the following steps: taking a sample of organic material by continuously maintaining a total microbiological, atmospheric and physical isolation between a sampling volume, the sample and a collection portion w herein the sample is confined, manipulating said sample under sterility conditions by an isolator apparatus, for determining a presence and/or an amount and/or developing capabilities of stem cells trapped in said sample and preserving the sample; the steps of taking the sample, manipulating the sample and preserving the sample are performed on a sample comprising at least a portion of organic tissue wherein stem cells are trapped in and are also performed on the sample in its entirety, without separating the stem cells from the portion of organic tissue.

The present invention relates to a method of collection, classification and preservation of samples containing stem cells in different operating fields, such as sampling and preservation of organic specimens in the clinical/laboratorial sphere.

It is known that in different production fields sampling of a substance presenting a complex structure and/or different aggregative states (solid or nearly-solid portions of tissue, mixed heterogeneously with fluids of micro-particles or peculiar types of cells or agglomerates and so on)from a given closed volume is made necessary, as it may happens in prenatal medical examples.

In particular, in the so-called CVS (i.e. Chorionic Villus Sampling), many operations have to be performed on a sample based on portions of aggregated tissue (that is, the chorionic villi) which exhibit a very compact structure, in complete contrast with the substantially liquid-phase samples taken for amniocentesis or blood spillages.

Presently, the known art does not contemplate any method involving long-term use of chorionic above all in connection with the possibility of preserving, taking out and subsequently manipulating the stem cells that can be found suspended therein.

In the application example briefly described above, the sample is to be maintained in an operating volume that must be as much as possible isolated from external agents (atmosphere, non-sterile environments, foreign substances and so on).

Generally, the known methods for operating/manipulating organic samples in heterogeneous state of aggregation have some drawbacks.

First, execution of known methods requires separation of the liquid phases from the solid (or tissutal) parts of the sample, so that analysis can be performed on a selected “portion” of the sample itself.

As a consequence of this, the need for separation of fluids from other materials (of organic or non-organic nature) implies high exposure to risks of contamination of the taken samples, with all negative consequences resulting therefrom.

In addition, referring in particular to organic samples exhibiting a mainly “solid” aggregation state and containing stem cells, implementation of a method enabling an efficient and accurate collection and preservation of same is hitherto unknown, in particular whenever there is a requirement for putting into practice the “working” and manipulation techniques that can (or could in the future) be applied to the stem cells themselves.

In the light of the above, the present invention aims at conceiving a collection, classification and preservation method that is able to obviate the set out drawbacks or in any case to overcome the operating limits of the operating protocols of the traditional type.

In particular, the present invention aims at conceiving a collection, classification and preservation method that is able to ensure a full isolation without interruption between the taken sample of fluid and/or material and the external/extracorporeal environment, both at the moment of the true sampling and during the subsequent periods of storage and preservation.

The present invention also aims at conceiving a method that can be used in the already known analytical sampling methods (both in the clinical/medical field and in other sectors), without said methods being drastically changed from the point of view of their operating sequences.

It is also an aim of the present invention to provide a method having high efficiency, high precision in terms of volume of the collected sample and/or material, low implementation costs and great ease in use.

From the point of view of the method, the present invention aims at conceiving a process enabling well precise volumes of “mainly solid-state” samples in a fully safe manner (as regards external contamination) and more quickly than with traditional methods.

Still more generally, the present invention aims at conceiving a method capable of sampling and preserving organic samples characterized by a prevalence of “solid-state” aggregation conditions (such as the material obtained when a CVS procedure is executed) and containing stem cells, so that it is possible to work on the stem cells therein contained and maintained in a preservation state, even after an indefinite period of storage.

Still as regards the method, the present invention wishes to make available a process enabling possible admixing of preserving substances to the sampled materials, in a short period of time and with the greatest reliability and accuracy (obviously while fully observing the requirement of isolating them from the external environment also during the step of adding a cryopreservation substance).

The foregoing and further aims are achieved by a method of collection, classification and preservation of organic samples, in particular samples containing stem cells, in accordance with the present invention, having the features shown in the appended claims and hereinafter illustrated in an embodiment thereof given by way of non-limiting example.

The method according to the invention can advantageously apply for collection, determination of quality/quantity/biological capabilities and preservation (as well as for a possible and subsequent new processing or culturing) of samples containing stem cells, and in particular of samples comprising the chorionic villi (that can be taken from a pregnant woman, for example).

It is to be noted in this connection that stem cells taken from the chorionic villi could be used as a cellular therapy source for treatment of pathologies in humans: in order to ensure use of these cells it is of the greatest importance:
to ensure sterility of the cellular product with which the patient will be re-inoculated;
to make sure that the stem cells to be re-inoculated have the necessary capabilities to develop into different human tissues; and
to make sure that viability and quantity of the stem cells to be re-inoculated are sufficient for a prompt and satisfactory reconstruction process.

Operatively, sterility of the chorionic villi sample during all the manipulation steps following true sampling, or possibly also the previously described preservation/storage step, is ensured by a dedicated laboratory instrument known in the technical field with the name of “isolator”: this particular isolator enables full separation between the operator (and above all any environmental alteration/pollution source connected with the operator's physical presence) and the sample of chorionic villi containing the stem cells.

From a structural standpoint, the isolator employable in the present method mainly comprises a work chamber which practically is a steel box completely isolated from the outside, provided with a filtering system having filters of the “Hepa” type and where access for the operator takes place through use of suitable gloves jutting out inside the box and sealingly connected with one of the box walls: conveniently, this box is pressurised to a greater pressure than the inlet chamber.

Also present is an inlet chamber (also termed “pass-box”) for introduction of the sample and the biological material to be processed, which chamber is provided with interlocking doors that do not allow direct communication between the work chamber and the surrounding environment.

Likewise, an outlet chamber is present which has a sterile sample-collecting bag: this outlet chamber is brought into communication with the work chamber by interlocking doors.

Finally, a sterilisation system is present for processing the biological sample and sterilising the outer surface of the biological-sample container; the sterilisation process takes place by use of hydrogen peroxide (H₂O₂) that is introduced into the isolator before each work step and between processing of two different biological samples in order to ensure absolute sterility during the manipulation step; during this step the “particle count” and/or “microbiological count” parameters will be continuously analysed.

As already stated before, and as it will be detailed herebelow, the present invention is related to a method of collection, classification and conservation of stem cells: this method basically comprises the following steps:
taking a sample of organic material by continuously maintaining a total microbiological, atmospheric and physical isolation between a sampling volume (e.g.: the area of a female patient wherein chorionic villi are available), the sample itself and a “collection portion” wherein the sample is confined, (e.g. a suitable sampler device or syringe);
manipulating said sample under sterility conditions by an isolator apparatus, at least for determining a presence and/or an amount and/or developing capabilities of stem cells trapped in the sample; and
preserving the sample.

Advantageously, the present method implies that the steps of taking the sample, manipulating the sample and preserving the sample are performed on a sample comprising at least a portion of organic tissue wherein stem cells are trapped in, and moreover the just cited steps (taking the sample and/or manipulating the sample and/or preserving the sample) are performed on the sample in its entirety, without separating the stem cells from the portion of organic tissue.

Surprisingly, the present method relies on the fact that despite chorionic villi stem cells are trapped in a medium substantially in a solid aggregation state (the tissue constituting the structure of the villi themselves), it has been found that qualitative/quantitative investigation of the properties/capabilities of the cells is still possible, and even the simultaneous conservation of the cells and of the “trapping medium” is possible without hampering the viability and the retrievability/separability of the cells from the medium whenever required.

As regards preserving (e.g. by freezing) of the stem cells, the following is done: at first, from about 20-25 mg of chorionic villi obtained through CVS (i.e. Chorionic Villus Sampling), an aliquot part of 3-5 mg will be taken.

Then the sample contained in 15 ml test tube with conical bottom and screw plug, is centrifuged at 2000 rpm for 10 minutes.

After centrifugation, the test tube is inserted into the isolator and without disturbing the so-called “cellular pellet”, the so-called “supernatant” is taken and preserved for preparing the freezing solution for the sample with final 10% DMSO.

This solution is cooled using a suitable cooling apparatus positioned inside the isolator and 1 ml thereof is used to suspend the so-called “cellular pellet” again, said pellet being then inserted into a suitable test tube for freezing (which instead has been caused to come out of the isolator's outlet chamber and then frozen by a programmable freezer).

At the end of the freezing step, the sample is preserved in suitable storage tanks operating with liquid nitrogen, containing liquid nitrogen fumes. Generally freezing is carried out on non-hematic samples and on samples non containing meconium that have been taken 24-48 hours earlier.

Defrosting of the stem cells is carried out by taking the sample out of the liquid nitrogen, positioning it in ice and bringing it into a 37° C. thermostat in the shortest period of time.

After about 3 minutes, the defrosted sample is transferred into the isolator and then drop-wise transferred into a 15 ml test tube with conical bottom and screw plug (this test tube contains about 9 ml of washing medium).

At the end of the addition, the test tube is caused to come out of the isolator's outlet chamber and is subsequently centrifuged at 1500 rpm for 10 minutes.

After centrifugation, the test tube is inserted into the isolator and without disturbing the chorionic villi fragments, the supernatant is taken; at this point, the cells contained in the chorionic villi fragments are suspended again with a suitable cell-growth substance (termed “medium”) in an amount of about 1-4 ml; the chorionic villi fragments are be disgregated through enzymatic reagents.

After this step, the enzymatic reagent will be removed and the cells obtained will be transferred in a suitable flask (in jargon termed “T25”) for expansion. It is to be noted that in accordance with the present invention, the method of collection and preservation of organic fluids and/or materials containing stem cells (such as chorionic villi that can be taken from a pregnant woman) can therefore comprise a cryogenic preservation step and also subsequent steps of thawing the cells.

More generally, it is also to be noted that the collection and preservation method applicable to organic fluids and/or materials containing stem cells can advantageous comprise a sampling step (carried out on a suitable “collection volume”) that is executed while a perfect microbiological, atmospheric and physical isolation is continuously maintained between the sampling volume, the sample of collected fluid and/or material and the collection portion wherein the fluid/material is confined.

As regards the above mentioned isolator, it is to be noted that within the scope of the present invention the structure of the latter has been suitably conceived for maximising the efficiency of the work method.

First of all, it will be appreciated that the size of the isolator's work chamber (which can be considered by way of example as a cubic volume having sides of about 80 cm) has been suitably studied so as to reduce the sizes of the inner surfaces and the volume: this geometric effect greatly reduces the time required for decontamination of the work chamber and also reduces consumption of sterilising agents (such as hydrogen peroxide) required for decontamination.

The isolator also offers the possibility of pre-cooling the freezing solution by using a thermo-block (made of steel) housed inside the work chamber: this positioning of the thermo-block ensures maximum sterility for the biological sample and the work area, unlike known systems exploiting a more traditional cooling by ice (which is not sterile and cannot be sterilised) contained in a container that in turn is not sterile.

The structural features of the isolator in this manner offer the possibility of keeping the biological sample inside the work chamber at a controlled temperature (i.e. temperature-regulating means is present in the work chamber, so that suitable comfort conditions can be achieved both for the product and the operator).

At the same time the isolator is provided with sealed and/or hermetic and/or sterile packaging means located at the outlet chamber: this sealed and/or hermetic and/or sterile packaging means offers the possibility of packaging the product coming out of the isolator in an aseptic manner, by a suitable sterile “rolled bag” (which in turn is useful for the purpose of carrying the sample to the cryogenic freezing and/or cryogenic preservation point).

In addition, due to the presence of the sterile “rolled bag” or equivalent means, possible working waste can also be eliminated without contaminating any part of the isolator (and therefore avoiding further sterilisation cycles being carried out).

The present isolator further has suitable filters (made of Gore-tex for example) that are operatively active on a line for admission of sterilising agents, and preferably a line for admission of hydrogen peroxide; in this manner a further lowering of the decontamination time is obtained.

In order to reach an ergonomic improvement for the operator, the isolator also has suitable positioning means for a biological sample (or in other words, for the sampled fluid and/or Material), as well as for the material required for processing: conveniently, this means can consist of a steel rack having the same surface finish degree as the isolator's walls.

The isolator can also be provided with self-governing movement means (a train of pivoting wheels or the like, for example) that allow displacement of same inside the room: practically, the self-governing movement means, allows the isolator to be shifted to the desired points, thus facilitating cleaning of the room and maintenance of the isolator itself, for example (or in any case offering the possibility of shifting the isolator to points in the room or laboratory that are more advantageous from an operating point of view).

As already said, the isolator also comprises sterilisation means acting at least on the work chamber and/or the inlet chamber and/or the outlet chamber: this means can conveniently atomise one or more sterilising agents so as to decontaminate every point of the isolator itself.

For completion of the different operating aspects of the isolator, suitable sensor means is then present which acts at least in the work chamber (but, if necessary, also in the inlet chamber and/or the outlet chamber), said sensor means being able to measure:
a microbiological sampling (typically, by an air intake positioned close to the work region, so as to have a truer and safer view of the working neighbourhood;
an air speed within the work chamber (these sensors can be possibly coupled with filtering means, comprising one or more filters of the “hepa” type for example, and/or with venting means acting on the work chamber and/or the inlet chamber and/or the outlet chamber);
a given number of particle values of the air during the whole process;
a possible residual amount of sterilising agents still in the work chamber after a sterilisation process.

Depending on the different occasions, the above described isolator can also be used separated from the method being the object of the present invention, i.e. in other industrial and/or laboratory processing methods on several different types of materials and/or (biological and non-biological) samples.

The invention enables achievement of important advantages.

In terms of operation, the present invention therefore enables accomplishment of a quicker collection, classification and preservation method as compared with traditional methods in use; this method also allows two operations that are generally carried out at different times (and therefore are time-consuming) to be integrated into a single operating step.

It will be appreciated that this method is in any case compatible with the already known operating methodologies and that a different degree of preparation by the staff putting it into practice is not required.

Finally, the present invention enables low implementation times and costs to be achieved both in terms of collection, classification and preservation device and in terms of cheap management of the sampling/storage/analysis works that are necessary in a great number of technological fields.

1. A method of collection, classification and conservation of stem cells, comprising the following steps:
taking a sample of organic material by continuously maintaining a total microbiological, atmospheric and physical isolation between a sampling volume, said sample and a collection portion wherein the sample is confined;
manipulating said sample under sterility conditions by an isolator apparatus, said manipulating step comprising at least a sub-step of determining a presence and/or an amount and/or developing capabilities of stem cells trapped in said sample; and
preserving the sample,
characterized in that the steps of taking the sample, manipulating the sample and preserving the sample are performed on a sample comprising at least a portion of organic tissue wherein stem cells are trapped in.

2. A method as claimed in claim 1, wherein the steps of taking the sample and/or manipulating the sample and/or preserving the sample are performed on the sample in its entirety, without separating said stem cells from said portion of organic tissue.

3. A method as claimed in claim 1, wherein said isolator apparatus comprises:
a work chamber isolated from the external environment and provided with a filtering system, an operator being able to accede to said work chamber by use of suitable gloves jutting out at the inside of the box and sealingly connected with one of the work chamber walls, said work chamber being pressurised to a greater pressure than the inlet chamber;
an inlet chamber for introduction of a sample and/or a biological material to be processed, said inlet chamber being connected to the work chamber and being provided with interlocking doors that do not allow direct communication between the work chamber and the external environment;
an outlet chamber connected with the work chamber and having a sterile sample-collecting bag, said outlet chamber being brought into communication with the work chamber by means of interlocking doors; and
a sterilisation system for processing the biological sample and sterilising an outer surface of a container of the biological sample itself, the sterilisation process taking place by use of hydrogen peroxide introduced into the isolator before each work step and between the operations for processing two different biological samples.

4. A method as claimed in claim 1, further comprising a step of continuously controlling a particle-count and/or microbiological-count parameters.

5. A method as claimed in claim 1, wherein said step of taking the sample further comprises the following sub-steps:
taking an amount of chorionic villi, preferably obtained through CVS (Chorionic Villus Sampling), said amount being comprised between 3 and 5 mg; and
admitting said amount of chorionic villi into a first container, said first container being preferably a 15 ml test tube with a conical bottom and a screw plug; and
centrifuging said amount of chorionic villi, said centrifuging sub-step being preferably carried out at 2000 rpm for 10 minutes.

6. A method as claimed in claim 5, wherein the step of manipulating the sample further comprises the following steps in sequence:
inserting said first container into the isolator; and
taking a supernatant from said first container.

7. A method as claimed in claim 1, wherein the step of preserving the sample further comprises the following sub-steps:
adding the sample with 10% DMSO (dimethyl sulfoxide);
cooling the sample, preferably by using a suitable freezing apparatus positioned within the isolator;
suspending chorionic villi fragments pellet formed by said sub-step of cooling the sample with 10% DMSO;
inserting said chorionic villi fragments pellet into a second container, said second container being preferably a test tube;
freezing said second container containing said chorionic villi fragments pellet;
extracting said second container through said outlet chamber of the isolator; and
freezing said second container by means of a programmable freezer.

8. A method as claimed in claim 1, further comprising a step of storing the sample in suitable storage containers, said storage containers preferably operating with liquid nitrogen and containing liquid nitrogen fumes.

9. A method as claimed in claim 1, further comprising a step of defrosting the sample, said step of defrosting the sample preferably comprising the following sub-steps:
taking the sample from liquid nitrogen;
positioning the sample in ice; and
bringing the sample into a thermostat at 37° C.

10. A method as claimed in claim 9, further comprising the following steps in sequence and after said step of defrosting the sample:
transferring the sample into the isolator into a third container, said third container being preferably a test tube having a 15 ml capacity, a conical bottom and a screw plug and more preferably containing 9 ml of a washing medium; and
centrifuging said third container after said transferring step, said centrifuging step being preferably carried out at 1500 rpm for 10 minutes;
inserting the third container into the isolator;
taking the surnatant, without disturbing the chorionic villi fragments, from said third container;
suspending cells contained in the chorionic villi fragments within a suitable cell-growth substance, said cell-growth substance amounting preferably from 1 to 4 ml;
disgregating the chorionic villi fragments through enzymatic reagents;
removing said enzymatic reagents; and
transferring the cells, said cells remaining after said disgregation of the chorionic villi fragments and after removal of the enzymatic reagents, and the cell-growth substance in a flask, said flask being preferably of type “T25”.
